(19) United States
(12) Patent Application Publication
(10) Pub. No.: US 2008/0241073 A1
(43) Pub. Date: Oct. 2, 2008

(54) Polymeric Micelle Type Mri Imaging Agent

(75) Inventors: Masayuki Yokoyama, Chiba (JP); Teruo Okano, Chiba (JP); Emiko Nakamura, Tokyo (JP)

Correspondence Address: BIRCH STEWART KOLASCH & BIRCH, PO BOX 747, FALLS CHURCH, VA, 22040-0747, US

(73) Assignee: Kanagawa Academy of Science and Technology, Kawasaki-shi (JP)

(21) Appl. No.: 11/631,527

(22) PCT Filed: Jan. 6, 2005
(86) PCT No.: PCT/JP2005/000058

§ 371 Date: Feb. 26, 2008

(30) Foreign Application Priority Data
Jul. 5, 2004 (JP) 2004-198464

(51) Int. Cl. A61K 49/12; C08G 69/48

(52) U.S. Cl. 424/9.322; 525/420

(57) ABSTRACT

A contrast agent for magnetic resonance imaging which stably circulates in the blood for a long period and which targets solid tumors, with which clear images of cancers may be obtained is disclosed. The contrast agent for magnetic resonance imaging comprises as an effective ingredient a polymeric micelle having gadolinium (Gd) atoms in an inner core and an outer shell including hydrophilic polymer chain segments, which micelle is delivered to a tissue(s) and/or site(s) of solid tumor(s) in vivo, whose micellar structure is dissociated after being accumulated in the tissue(s) and/or site(s).

### TECHNICAL FIELD

The present invention relates to a contrast agent for magnetic resonance imaging, more particularly, to a contrast agent comprising as an effective ingredient a gadolinium (Gd)-containing polymeric micelle.

### BACKGROUND ART

Therapies for cancer are largely classified into three groups, that is, surgical treatment, radiotherapy and chemotherapy. Although the effective rate and cure rate continue to increase by virtue of the developments in the therapies, the developments cannot keep up with the increase in the incidence to allow increase in the death rate. What is common to these therapies is that early detection largely improves the treatment results. Therefore, developments in diagnostic techniques can greatly contribute to the decrease in death rate.

Diagnostic techniques of cancer include histological diagnoses of collected cells, biochemical tests of blood, diagnostic imaging and the like. Diagnostic imaging include X-ray CT, magnetic resonance imaging (hereinafter referred to as “MRI” for short), ultrasound imaging and the like. Among these, MRI is characterized in that it is free from X-ray exposure and is noninvasive, and that a high resolution next to X-ray CT is obtained.

For the purpose of improving accuracy of diagnosis, MRI contrast agents are used. After administering an MRI contrast agent, images are taken by MRI. As the MRI contrast agents, low molecular chelating compounds to which a Gd atom(s) coordinate(s) are frequently used. A representative example of such a complex is Gd-DTPA commercially available under the trademark Magnevist (DTPA means diethylenetriaminepentaacetic acid which is a low molecular chelating agent, wherein one Gd atom coordinates to one molecule of DTPA). The Gd atom in this chelating agent acts on the hydrogen atoms in water molecules existing in the vicinity thereof to shorten the T1 (longitudinal relaxation time) thereof. By appropriately setting the various device parameters in MRI measurement, the water molecules having a shortened T1 can be clearly distinguished from other water molecules in the image. Thus, by virtue of the T1-shortening effect, a high contrast is obtained in the MRI image. Gd-DTPA mainly images the blood with a high contrast to clearly show abnormal angiogenesis in tumor tissues, thereby serving for diagnostic imaging. Thus, Gd-DTPA per se does not have a selectivity to solid tumors. Since Gd-DTPA is low molecular so that its permeation from the blood vessels to a tissue is quick, MRI imaging must be started immediately after injection of the contrast agent to the body. Thus, in case of, for example, the patient is suddenly indisposed and the patient has a rest for about 2 hours, injection of the MRI contrast agent must be done again.

Aiming at improving the above-mentioned drawback of the low molecular MRI contrast agents and developing a contrast agent with a higher performance, studies to bind Gd atoms having MRI effect to a polymer have been made since 1980's. These studies mainly aim at targeting the contrast agent to solid tumor and the like due to the property of the polymer so as to obtain an MRI image selective to the target, thereby serving for more accurate diagnosis of the disease, and also aim at extending the time range after administration of the contrast agent, in which appropriate imaging can be attained utilizing the fact that the diffusion rates of the polymeric contrast agents are lower than those of the low molecular contrast agents, thereby making MRI diagnosis easier for both the patients and physicians.

Representative examples of the polymeric MRI contrast agents include those using albumin or polysaccharide derivatives which are natural polymers, and those using poly(L-lysine) derivatives. More specifically, there are the following 3 examples: Wikstrom et al, have reported an MRI contrast agent in which a plurality of DTPA molecules, which is a chelating agent, to albumin and Gd atoms are coordinated to the DTPA (Non-patent Literature 1). By virtue of the fact that Gd atoms are bound to albumin which is polymeric substance, the T1-shortening ability (called relativity) per one Gd atom is increased to about 4 times that of the low molecular Gd-DTPA. It is understood that the increase in the relaxivity is brought about by the fact that the movement of Gd atom is restricted by being bound to a polymeric substance. The increase in the relaxivity is one of the advantageous features of polymeric MRI contrast agents. Corot et al. have reported a polymeric MRI contrast agent in which DOTA (tetraazacyclododecanetetraacetic acid) which is a chelating agent is bound to carboxymethyldextran which is a polysaccharide, and Gd atoms are coordinated thereto (Non-patent Literature 2). In this example too, by constituting the contrast agent with a polymeric substance, the relaxivity of T1 was increased such that the T1 relaxivity of the polymeric MRI agent was 10.6 which was about 3 times that of the low molecular counterpart, DOTA-Gd, that was 3.4. In this study, change in the plasma level of the contrast agent after administration thereof to rats was also observed. It is reported that little more than 40% of the administered amount of the contrast agent was present in the plasma at 30 minutes after the intravenous administration. Although this level was about 5 times higher than that of the corresponding low molecular contrast agent, DOTA-Gd, the circulating property is thought to be still insufficient for targeting or being delivered to solid tumor.

The study which best attained the selective targeting (or delivery) to solid tumor by optimizing the structure of the polymer to attain stable circulation in the blood for a long period is the study by Weissleder et al (Non-patent Literature 3). They succeeded in making the contrast agent circulate in the blood for a long period and in targeting of Gd coordinated to DTPA to solid tumor by using, as a carrier, a polymer in which polyethylene glycol chains are bound to poly(L-lysine) (The accumulated amount in a solid tumor at 24 hours after administration to a rat weighing about 150 g was about 1.5% dose/g). However, even in this study, obtaining a clear image of a tumor did not succeed.

### Non-patent Literature 1: Investigative Radiology, 24, 609-615 (1989)

Non-patent Literature 2: Acta Radiologia, 38, supplement 412, 91-99 (1997)

### Non-patent Literature 3: J. Drug Targeting, 4, 321-330 (1997)

### DISCLOSURE OF THE INVENTION

### Problems which the Invention Tries to Solve

An object of the present invention to provide a contrast agent which stably circulates in the blood for a long period of time and which targets solid tumor, by which clear image of the tumor may be obtained.

### Means for Solving the Problems

The present inventors presumed that the main reason why a clear image is not necessarily obtained by using the contrast agent by Weissleder et al. is that not only the vessels in the tumor tissue, but also the vessels in normal tissues are imaged with high contrast. More specifically, the present inventors presumed as follows: Since the rate of transfer of a polymeric substance from the blood flow to a tumor tissue is low, in general, in order to transfer a large amount of the polymeric substance to the tumor tissue, the contrast agent system must be designed such that it can circulate in the blood for a long period of time so that the contrast agent obtains much chance to transfer. On the other hand, with such a contrast agent system, a large amount of the contrast agent remains in the blood vessels in the normal tissues even after the contrast agent has been sufficiently targeted to the tumor tissue(s), so that a large difference in the signal intensity between the normal tissues and the tumor tissue(s) cannot be obtained.

Based on this presumption, the present inventors have studied for providing a Gd-polymer conjugate which is likely to dissociate in the tumor tissue(s) or a cancer site(s), while which can stably keep, to some degree, the state where the Gd atoms are blocked in the blood flow in the normal vessels. As a result, the present inventors discovered that a certain Gd-containing polymeric micelle can attain the above-described object.

Thus, according to the present invention, a polymeric micelle having gadolinium (Gd) atoms in an inner core and an outer shell including hydrophilic polymer chain segments, which micelle is delivered to a tissue(s) and/or site(s) of solid tumor(s) in vivo, whose micellar structure is dissociated after being accumulated in the tissue(s) and/or site(s) of the solid tumor(s) is provided, and an MRI contrast agent comprising such a polymeric micelle as an effective ingredient is provided.

Preferred modes of the present invention include the above-described polymeric micelle and the contrast agent, wherein the polymeric micelle is formed of a block copolymer(s) having a hydrophilic polymer chain segment and a polymer chain segment having carboxyl groups and residues of a chelating agent(s) in its(their) side chains, gadolinium atoms coordinated to the block copolymer(s), and a polyamine(s).

More preferred modes of the present invention include the above-described polymeric micelle and use thereof as an MRI contrast agent, wherein the above-described block copolymer is poly(ethylene glycol)-block-poly(aspartic acid) and said residues of a chelating agent(s) are introduced to 5% to 30% of the recurring units of the aspartic acid.

According to another mode of the present invention, a specific block copolymer which can form the above-described polymeric micelle is also provided.

### EFFECTS OF THE INVENTION

According to the present invention, by using the above-described polymeric micelle as an MRI contrast agent, the T1 relativity of the water in the vessels in normal tissues and that of the water in the solid tumor tissue(s) may be clearly distinguished. That is, the polymeric micelle according to the present invention (formed by association of several hundreds of polymer molecules, having bilayer structure including an inner core and an outer shell, wherein Gd atoms are coordinated in the core), used as a nano-sized carrier system, selectively transports (targets) the Gd atoms that make the contrast in MRI images to the solid tumor locally, thereby enabling to clearly image microcarcinoma, which hitherto could not be attained by the conventional MRI cancer diagnosis systems. The Gd atoms act on the hydrogen atoms in water molecules existing in the vicinity thereof to shorten the T1 (longitudinal relaxation time) thereof. By virtue of the T1-shortening effect, a high contrast is obtained in the MRI image.

Although not bound by a theory, the reason why the polymeric micelle according to the present invention can target the solid tumor and why the polymeric micelle according to the present invention gives a high MRI contrast to the solid tumor tissue(s) is understood as follows: That is, the blood vessels constituting solid tumor tissue(s) have characteristics that they have an abnormally increased permeability to polymers and nano-sized particles, and they lack lymphatic capillary which is an elimination pathway of polymeric materials transferred to the normal tissues from the blood. Because of these characteristics, polymers and nano-sized particles selectively accumulate in the tumor tissue(s), that is, targeted to the tumor tissue(s). This effect is known as EPR effect (Enhanced Permeability and Retention effect) (see Matsumura, Y. et al., Cancer Res., 46, 6387-6392 (1986)). It is a great advantage that merely a polymer or nano-sized particles whose surface does not adhere to the cells is required to exhibit EPR effect, and a specific antibody or the like to the cancer cells is not necessary. By virtue of this effect, it has been shown that the polymer or particles may be targeted to the tumor tissue(s) at a level of 3 to 10 times higher than in the level of normal tissue(s) in various examples. An example of the particles targeting an anti-cancer agent to solid tumor include the polymeric micelle system containing an anti-cancer agent, adriamycin, which was developed by Yokoyama and Okano et al., who are also the co-inventors of the present application (see M. Yokoyama, et al., J. Drug Targeting, 7(3), 171-186 (1999)).

There is another design for selectively giving a high contrast to the solid tumor in MRI images, other than the above-described targeting effect. It is the change in T1-shortening ability based on the formation and dissociation of the micellar structure. The concept of delivery of the polymeric micelle to the solid tumor through circulation in the blood is shown in FIG. 1. As shown in FIG. 1, when the micelle has the polymeric micellar structure during the circulation in the blood, Gd atoms are located within the inner core of the micelle and are separated from the water molecules outside, so that they cannot sufficiently exert their ability to shorten T1 of the water molecules. That is, when the polymeric micellar structure is kept, increase in the MRI contrast does not occur. On the other hand, the polymeric micelle delivered to a tumor tissue gradually dissociate into a Gd-bound block copolymer and a positively charged polymer. In this dissociated state, Gd atoms can access to the water molecules, so that they exert the T1-shortening ability to give a high contrast to the tumor tissue. Moreover, it is known that the T1-shortening ability per atom of the Gd atoms bound to the polymer is increased to twice to three times that of free Gd atom because of the effect that the movement of Gd atoms bound to the polymer is restricted by the polymer. Even if micellar structure is dissociated during the circulation in the blood, the dissociated block copolymer is quickly excreted into the urine by the filtering action of the kidney, so that it does not give a high contrast to the blood. On the other hand, in the tumor tissue, it is thought that even the dissociated Gd-coordinated block copolymer has a sufficient size to be retained in the tissue, so that it remains in the tissue for a long period of time to continuously give a high MRI contrast.

### BRIEF DESCRIPTION OF THE DRAWINGS

FIG. 1 shows the concept of delivery of the polymeric micelle to a solid tumor through circulation in the blood.

FIG. 2 schematically shows a production process and the structure of a preferred example of the polymeric micelle according to the present invention.

### BEST MODE FOR CARRYING OUT THE INVENTION

Each constituent of the present invention will now be described in detail. The micelle according to the present invention is an aggregate of molecules formed by association of several hundreds of polymer molecules in an aqueous medium, which micelle has a bilayer structure including an inner core and an outer shell, wherein Gd atoms are coordinated in the core. Since the polymeric micelle is delivered to and accumulated in a solid tumor tissue(s) in the body (e.g., in the body of a mammal such as human), the polymeric micelle is in the form of nano-sized ultrafine particles having a diameter of, for example, about 10 nm to 100 nm. The behavior that “the micellar structure is dissociated after being accumulated in solid tumor tissue(s)” may be confirmed by examining whether the polymeric micelle is dissociated or not in an aqueous solution having a sodium chloride concentration higher than that in the blood, which solution can be an in vitro model of the solid tumor.

The polymer which forms such a micelle may be any block copolymer as long as it is a block copolymer having a hydrophilic polymer chain segment and a polymer chain having side chains to which Gd may be coordinated, which block copolymer can form the above-described nano-sized ultrafine particles in an aqueous medium in the presence of a polyamine, and which block copolymer can be dissociated to decompose the micellar structure. Therefore, the hydrophilic polymer chain segment forming the outer shell may be any water-soluble polymer as long as it is suited for the purpose of the present invention. Although not restricted, the block copolymer includes a polymer chain segment derived from polyethylene glycol, poly(vinylalcohol) and poly(vinyl pyrrolidone). Similarly, the polymer chain segment which is another segment in the block copolymer, which forms the inner core to which Gd may be coordinated thereby fixing Gd may be any one derived from a polymer having side chains to which Gd may be effectively coordinated. Specific examples of such a polymer chain segment includes those derived from poly(aspartic acid), poly(glutamic acid), poly(acrylic acid) and poly(methacrylic acid), wherein residues of a chelating agent(s) are introduced to a prescribed percentage of the carboxyl groups in the recurring units.

Thus, specific examples of the block copolymer which may be used in the present invention include polyethylene glycol-block-poly(aspartic acid), polyethylene glycol-block-poly(glutamic acid), polyethylene glycol-block-poly(acrylic acid), polyethylene glycol-block-poly(methacrylic acid), polyvinyl alcohol)-block-poly(aspartic acid), poly(vinyl alcohol)-block-poly(aspartic acid), polyvinyl alcohol)-block-poly(glutamic acid), poly(vinyl alcohol)-block-poly(acrylic acid), poly(vinyl alcohol)-block-poly(methacrylic acid), poly(vinyl pyrrolidone)-block-poly(aspartic acid), poly(vinyl pyrrolidone)-block-poly(glutamic acid), poly(vinyl pyrrolidone)-block-poly(acrylic acid) and poly(vinyl pyrrolidone)-block-poly(methacrylic acid), to which residues of a chelating agent(s) are bound by covalent bond through carboxyl groups of the block copolymer and through linkers, as required. The term “block copolymer” is meant to include those wherein one or both ends of the polymer chain are modified so as to be able to bind another functional molecule such as an antibody, antigen, hapten or the like (see the X group in the formulae below). Many of the block copolymers per se to which the residues of the chelating agent(s) are not bound are known, and even if the block copolymer is novel, it may be produced by a method which per se is known, for example, by the method described in U.S. Pat. No. 5,449,513 (JP-A-6-107565) in the case of block copolymers having a poly(amino acid) segment. Those having a poly(meth)acrylic acid segment may be obtained by Atomic Transfer Radical Polymerization described in K. Matyjaszawski et al., Chem. Rev., 101, 2921-2990 (2001).

The molecular weight of the hydrophilic polymer chain segment in the block copolymer, such as polyethylene glycol moiety, is preferably about 2000 to 20,000, more preferably about 4000 to 12,000.

Specific examples of the linker include —NH(CH₂)ₙ—NH (wherein n represents an integer of 1 to 6), that is, ethylenediamine(—NHCH₂CH₂NH—), hexamethylenediamine (—NH(CH₂)₆NH—) and the like.

The residue of the chelating agent may be one originated from a chelating agent selected from the group consisting of diethylenetriaminepentaacetate (DTPA), tetraazacyclododecane (DOTA), 1,4,7-tris(carboxymethyl)-10-(2′-hydroxypropyl)-1,4,7,10-tetraazacyclododecane (DO3A) and the like, although the residue of the chelating agent is not restricted thereto as long as it is suited for the object of the present invention. Needless to say, the chelating agent is bound to the above-described linker or an oxygen atom such that a moiety other than the group required for the chelating is bound to the linker or the oxygen atom so that it can chelate a gadolinium atom(s).

In cases where the residues of the chelating agent(s) are bound through linkers, the percentage of the linkers which remain unbound to the residue of the chelating agent may preferably be as small as possible, and the percentage of the free linkers is preferably not more than ½, more preferably not more than ⅓ of the total linkers.

Examples of the block copolymer which may preferably be used in the present invention include polyethylene glycol-block-poly(aspartic acid), polyethylene glycol-block-poly(glutamic acid) in which the residues of the chelating agent(s) are introduced to a prescribed amount of carboxyl groups. Taking these as examples, block copolymer will now be described in more detail.

These block copolymers are, more particularly, represented by the following formulae (1_{A-1}), (1_{A-2}), (1_{B-1}), (1_{B-2}), (1_{C-1}), (1_{C-2}), (1_{D-1}) and (1_{D-2}), respectively:

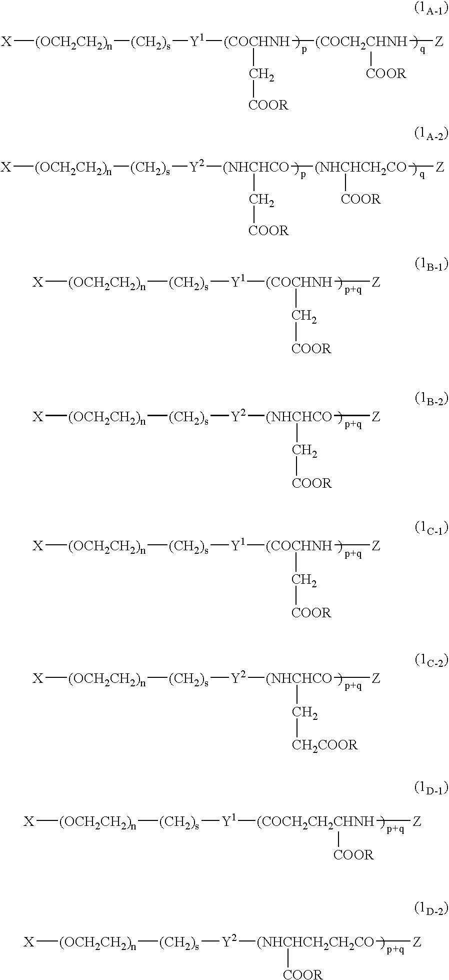

In each of the above formulae, X represents hydrogen, C₁-C₆ alkyl, hydroxy-C₁-C₆ alkyl, acetalized or ketalized formyl-C₁-C₆ alkyl, amino-C₁-C₆ alkyl or benzyl;

Z represents hydrogen, hydroxy, C₁-C₆ alkyl, C₁-C₆ alkyloxy, phenyl-C₁-C₄ alkyl, phenyl-C₁-C₄ alkyloxy, C₁-C₄ alkylphenyl, C₁-C₄ alkylphenyloxy, C₁-C₆ alkoxycarbonyl, phenyl-C₁-C₄ alkyloxycarbonyl, C₁-C₆ alkylaminocarbonyl or phenyl-C₁-C₄ alkylaminocarbonyl;

n represents an integer of 10 to 10,000;

s represents an integer of 0 to 6;

OR represents OH, a linker preferably —NHCH₂CH₂NH₂) or a residue of a linker-chelating agent (preferably —NHCH₂CH₂NHCOCH₂(HOOCH₂—)—NCH₂CH₂N(CH₂CH₂COOH)—CH₂CH₂N—(CHCHCOOH)₂), wherein the number of the residues of the chelating agent(s) is 5 to 30% of the total of p+q; p and q independently represent integers of 1 to 300;

Y¹ represents —NH— or R^{a}—(CH₂)ᵣ—R^{b}— wherein R^{a} represents OCO, OCONH, NHCO, NHCONH, COO or CONH, and R^{b} represents NH or O; and Y² represents CO or —R^{c}—(CH₂)ᵣ—R^{d}— wherein R^{c} represents OCO, OCONH, NHCO, NHCONH, COO or CONH, R^{d} represents CO, and r represents an integer of 1 to 6.

Since the number of the residues of the chelating agent(s) is 5 to 30% of the total of p+q, p+q is not less than 4, of course.

As the block copolymer, those represented by the following Formula (2) may also preferably be employed:

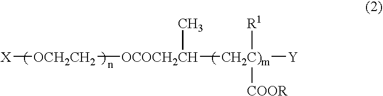

(wherein X represents hydrogen, C₁-C₆ alkyl, hydroxy-C₁-C₆ alkyl, acetalized or ketalized formyl-C₁-C₆ alkyl, amino-C₁-C₆ alkyl or benzyl; R¹ represents hydrogen or methyl; Y represents hydrogen, OH, Br, OR², CN, OCOR², NH₂, NHR² or N(R²)₂ (wherein R² represents ______); m represents an integer of 4 to 600; OR represents OH, a linker or a residue of a linker-chelating agent, wherein the number of the residues of the chelating agent(s) is 5 to 30% of the m).

These block copolymers may be used as the block copolymer for forming the polymeric micelle according to the present invention. Moreover, to the best knowledge of the present inventors, these block copolymers are compounds which are not described in prior art references. Therefore, according to the present invention, the block copolymers represented by the above-described formulae (1_{A-1}), (1_{A-2}), (1_{B-1}), (1_{B-2}), (1_{C-1}), (1_{C-2}), (1_{D-1}) and (1_{D-2}), respectively, are also provided.

The alkyl moieties in C₁-C₆ alkyl or C₁-C₆ alkyloxy or the like used in the present invention means those having 1 to 6 carbon atoms, such as methyl, ethyl, n-propyl, iso-propyl, n-butyl, tert-butyl and n-hexyl. The bonds or the linkers in the formulae described in the present specification are understood to bind the group, segment or the block in the direction shown in the formulae.

The above-described block copolymers having the residues of the chelating agent(s) may be conveniently produced according to the following reaction scheme, and then Gd may be coordinated thereto. It should be noted that although the reaction scheme below shows an example of the production process of a preferred block copolymer, other block copolymers may be produced by a similar method. Further, since each step per se in the reaction scheme below may easily be carried out by those skilled in the art based on common chemical knowledge, and since the conditions are described in the Examples below in detail, the process may easily be carried out based on the description of the Examples.

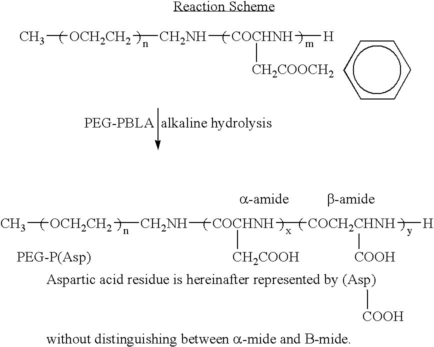

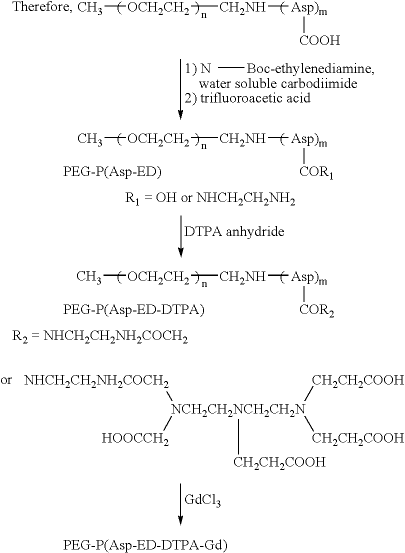

### Formation of Polymeric Micelle

The polymeric micelle may be prepared by preparing a mixed aqueous solution containing the Gd-carrying block copolymer obtained as described above and the polyamine in amounts such that the ratio of the carboxyl groups (—COOH) of the block copolymer to the amino groups (—NH₂) of the polyamine is adjusted to be 1:5 to 5:1, preferably 1:2 to 2:1; stirring the solution for several minutes to several hours at room temperature, or under warming or cooling, if required, after adjusting the pH to 6.5 to 7.5, if required; and dialyzing the resulting solution against distilled water using a dialysis membrane having a molecular weight cutoff of 1000. To the mixed aqueous solution, a water-miscible organic solvent(s), such as dimethylsulfoxide (DMSO) and/or N,N-dimethylformamide (DMF) ethyl alcohol may be added. The polyamine used in the present invention may be of any type and may have any molecular weight as long as it can form the polymeric micelle with the above-described block copolymer. Examples of the polyamine which may preferably be used include, but not limited to, poly(L-lysine), poly(D-lysine), poly(L-arginine), poly(D-arginine), chitosan, spermine, spermidine, polyallylamine and protamine. Those polyamines having a molecular weight of 500 to 50,000 may preferably be employed.

An example of the production process and structure of the polymeric micelle described above is schematically shown in FIG. 2.

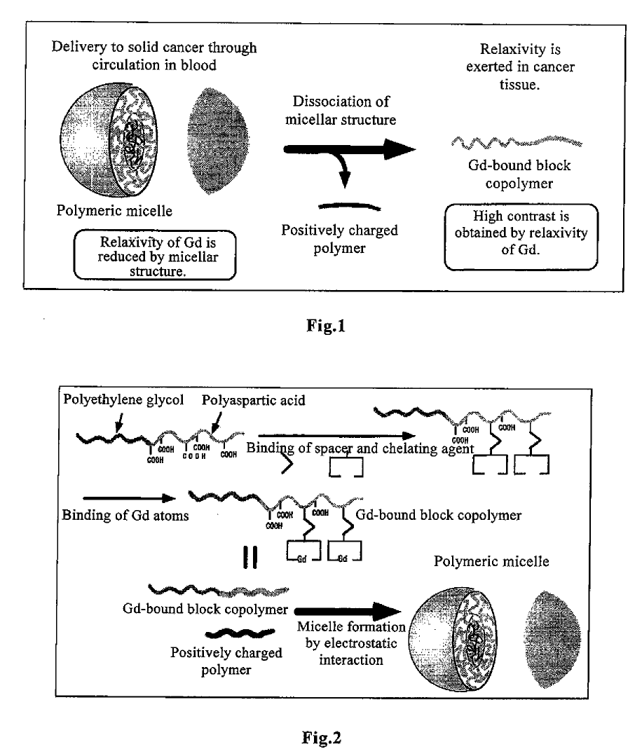

The thus obtained polymeric micelle exhibits the above-described actions and effects, and the actions and effects are schematically shown in FIG. 1 as mentioned above.

The present invention will now be described more concretely by way of Examples. These Examples are presented for the purpose of easier comprehension of the present invention.

### EXAMPLE 1

### Production of Block Copolymer having Residues of Chelating Agent

### (1) Alkaline Hydrolysis

To 1.00 g of polyethylene glycol-block-poly(β-benzyl L-aspartate) (hereinafter referred to as “PEG-PBLA” for short) whose polyethylene glycol moiety had a molecular weight of 5000 and whose β-benzyl L-aspartate moiety had a degree of polymerization of 44, was added 0.5N aqueous sodium hydroxide solution in an amount of 3.0 times by molar equivalent the β-benzyl L-aspartate units, and the resulting solution was stirred at room temperature for 15 minutes. When the solution became transparent, 6N hydrochloric acid in an amount of 10 times by molar equivalent the β-benzyl L-aspartate units was added. Thereafter, the resulting mixture was dialyzed against 0.1N hydrochloric acid and then against distilled water. Finally, the mixture was lyophilized to obtain polyethylene glycol-block-poly(aspartic acid) (hereinafter referred to as I“PEG-P(Asp)” for short). It has been confirmed that by this alkaline hydrolysis, about 75% of the main chain of the poly(aspartic acid) moiety of the polyethylene glycol-block-poly(aspartic acid) is β-amidated, and that decomposition of the main chain of the block copolymer does not occur.

In the same manner as described above, 3 types of PEG-P(Asp) shown in Table 1 below were obtained.

TABLE 1
Synthesis of PEG-P(Asp) Block Copolymer

| | | Molecular | Number of Aspartic |
|---|---|---|---|
| Run | Code | Weight of PEG | Acid (Asp) Units |
| 1 | 5000-26 | 5,000 | 26 |
| 2 | 5000-44 | 5,000 | 44 |
| 3 | 12000-26 | 12,000 | 26 |
| 4 | 12000-49 | 12,000 | 49 |

### (2) Binding of Ethylenediamine (ED) Units

In 7.8 mL of dimethylsulfoxide, 391 mg of PEG-P(Asp) whose polyethylene glycol moiety had a molecular weight of 5000 and in which the number of aspartic acid units was 44 was dissolved, and 144 mg of N-Boc-ethylenediamine and 166 mg of water-soluble carbodiimide were added, followed by stirring the mixture at room temperature for 4 hours. The reaction solution was dialyzed against distilled water using a dialysis membrane having a molecular weight cutoff of 1000, and the resulting solution was lyophilized to recover the polymer. The thus obtained polymer was then dissolved in trifluoroacetic acid and the resulting solution was stirred at 0° C. for 1 hour to eliminate the Boc groups. Thereafter, the reaction solution was dialyzed against distilled water using a dialysis membrane having a molecular weight cutoff of 1000, and the resulting solution was lyophilized to recover the polymer. The number of the introduced ethylenediamine units was measured by ¹H-NMR, which was 16.

In the same manner as described above, 10 types of PEG-P(Asp-ED) shown in Table 2 below were obtained.

TABLE 2
Synthesis of PEG-P(Asp-ED) Block Copolymer

| | | | | Number of |
|---|---|---|---|---|
| | | Molecular | Number of | Ethylenediamine |
| Run | Code | Weight of PEG | Asp Units | (ED) Units |
| 1 | 5000-26-5 | 5,000 | 26 | 5 |
| 2 | 5000-44-9 | 5,000 | 44 | 9 |
| 3 | 5000-44-13 | 5,000 | 44 | 13 |
| 4 | 5000-44-16 | 5,000 | 44 | 16 |
| 5 | 5000-44-22 | 5,000 | 44 | 22 |
| 6 | 12000-26-6 | 12,000 | 26 | 6 |
| 7 | 12000-26-7 | 12,000 | 26 | 7 |
| 8 | 12000-26-10 | 12,000 | 26 | 10 |
| 9 | 12000-49-13 | 12,000 | 49 | 13 |
| 10 | 12000-49-19 | 12,000 | 49 | 19 |

### (3) Binding of DTPA (Dithylenetriaminepentaacetic Acid) Units

In dimethylsulfoxide, 100 mg of PEG-P(Asp-ED)5000-44-9 (Run 2 in Table 2) was dissolved, and triethylamine in an amount of 1.5 times by molar equivalent the ethylene diamine residues and DTPA anhydride in an amount of 5 times by molar equivalent the ethylene diamine residues were added, followed by stirring the resulting mixture at room temperature for 1 day. The obtained solution was dialyzed against water, and the resulting solution was lyophilized. The number of the introduced DTPA units in the thus obtained DTPA-introduced block copolymer(PEG-P(Asp-ED-DTPA) was measured by ¹H-NMR, which was 6.

In the same manner as described above, 9 types of PEG-P(Asp-ED-DTPA) shown in Table 3 below were obtained.

TABLE 3
Synthesis of PEG-P(Asp-ED-DTPA) Block Copolymer

| | | Molecular | Number | Number | Number |
|---|---|---|---|---|---|
| | | Weight | of Asp | of ED | of DTPA |
| Run | Code | of PEG | Units | Units | Units |
| 1 | 5000-26-5-4 | 5,000 | 26 | 5 | 4 |
| 2 | 5000-44-9-5 | 5,000 | 44 | 9 | 5 |
| 3 | 5000-44-9-6 | 5,000 | 44 | 9 | 6 |
| 4 | 5000-44-9-7 | 5,000 | 44 | 9 | 7 |
| 5 | 5000-44-16-7 | 5,000 | 44 | 16 | 7 |
| 6 | 5000-44-16-9 | 5,000 | 44 | 16 | 9 |
| 7 | 12000-26-6-4 | 12,000 | 26 | 6 | 4 |
| 8 | 12000-26-7-5 | 12,000 | 26 | 7 | 5 |
| 9 | 12000-26-10-4 | 12,000 | 26 | 10 | 4 |

### EXAMPLE 2

### Binding of Gd

### Gadolinium Atoms

In 1.5 mL of distilled water, 20 mg of PEG-P(Asp-ED-DTPA)5000-44-16-9 (Run 6 in Table 3) was dissolved, and Gd in an amount of 2.0 times by molar equivalent the DTPA residues, in the form of aqueous GdCl₃ solution, was added thereto, followed by stirring the resulting mixture at room temperature for 15 minutes. An equal amount of EDTA (ethylenediaminetetraacetic acid) by molar equivalent to the carboxyl groups in the block copolymer was added to the resulting mixture, and the resultant mixture was stirred for 10 minutes. The resulting mixture was dialyzed against distilled water using a dialysis membrane having a molecular weight cutoff of 1000, and the resulting solution was lyophilized to recover the polymer. The amount of introduced Gd was determined using an ICP (Inductively Coupled Plasma) emission spectrophotometer, which was 7.

In the same manner as described above, 17 types of PEG-P(Asp-ED-DTPA-Gd) shown in Table 4 were obtained.

TABLE 4
Synthesis of PEG-P(Asp-ED-DTPA-Gd) Block Copolymer

| | | Molecular | Number | Number | Number | Number |
|---|---|---|---|---|---|---|
| | | Weight | of Asp | of ED | of DTPA | of Gd |
| Run | Code | of PEG | Units | Units | Units | Units |
| 1 | 5000-26-5-4-4 | 5,000 | 26 | 5 | 4 | 4 |
| 2 | 5000-26-5-4-3 | 5,000 | 26 | 5 | 4 | 3 |
| 3 | 5000-26-5-4-2 | 5,000 | 26 | 5 | 4 | 2 |
| 4 | 5000-44-9-5-3 | 5,000 | 44 | 9 | 5 | 3 |
| 5 | 5000-44-9-6-7 | 5,000 | 44 | 9 | 6 | 7 |
| 6 | 5000-44-9-7-15 | 5,000 | 44 | 9 | 7 | 15 |
| 7 | 5000-44-16-7-4 | 5,000 | 44 | 16 | 7 | 4 |
| 8 | 5000-44-16-9-6 | 5,000 | 44 | 16 | 9 | 6 |
| 9 | 5000-44-16-9-7 | 5,000 | 44 | 16 | 9 | 7 |
| 10 | 12000-26-6-4-5 | 12,000 | 26 | 6 | 4 | 5 |
| 11 | 12000-26-6-4-4 | 12,000 | 26 | 6 | 4 | 4 |
| 12 | 12000-26-6-4-2 | 12,000 | 26 | 6 | 4 | 2 |
| 13 | 12000-26-7-5-6 | 12,000 | 26 | 7 | 5 | 6 |
| 14 | 12000-26-10-4-6 | 12,000 | 26 | 10 | 4 | 6 |
| 15 | 12000-26-10-4-4 | 12,000 | 26 | 10 | 4 | 4 |
| 16 | 12000-26-10-4-3 | 12,000 | 26 | 10 | 4 | 3 |
| 17 | 12000-26-10-4-2 | 12,000 | 26 | 10 | 4 | 2 |

### EXAMPLE 3

### Formation of Polymeric Micelle by Block Copolymer and Polycation Polymer

The PEG-P(Asp-ED-DTPA-Gd) and the polycation polymer were separately dissolved in 0.5M aqueous NaCl solution, respectively, and the pH of the solutions was adjusted to 6.8 to 7.2. These solutions in equal amount were mixed and the mixture was stirred at room temperature for 15 minutes, followed by dialysis against distilled water using a dialysis membrane having a molecular weight cutoff of 1000. The thus obtained solution and 2-fold dilution of PEG-P(Asp-ED-DTPA-Gd) were subjected to the following measurements:

(1) Gel permeation chromatography
(2) Dynamic light scattering
(3) Measurement of T1 (longitudinal relaxation time) of water by ¹H-NMR
a) First, formation of micellar structure was confirmed by gel permeation chromatography.

Table 5 shows the results of mixing polyallylamine with an average molecular weight of 15,000 and a PEG-P(Asp-ED-DTPA-Gd) block copolymer. The elution volume of the block copolymer was larger than 6.2 mL was obtained, and the elution volume of the polyallylamine was 10 mL. Therefore, if an elution volume smaller than 6.2 mL, it is seen that polymeric micellar structure was formed. As shown in Table 5, two types of the block copolymer were respectively mixed with the polyallylamine at a charge ratio of 0.5, 1.0 or 2.0, and the elution volume was smaller than 6.2 mL in all cases, so that it was proved that micellar structure was formed. The average particle size of the polymeric micelle of Run 2 was measured by a dynamic light scattering apparatus, which was 55 nm. Among the charge ratios tested, the elution volume was the smallest and so the most stable micellar structure was formed when the charge ratio was 2.0 with any of the block copolymers. Therefore, the tests hereinafter were carried out at a charge ratio of 2.0

TABLE 5
Micelle Formation from PEG-P(Asp-ED-DTPA-Gd)
and Polyallylamine (PAA)

| | Structure of PEG-P | | Elution Volume (mL) |
|---|---|---|---|
| | (Asp-ED-DTPA-Gd) | Charge Ratio | in Gel Permeation |
| Run | (code) | —NH₂/—COOH | Chromatography |
| 1 | 5000-44-16-9-7 | 0.5 | 4.0 |
| 2 | 5000-44-16-9-7 | 1.0 | 5.6 |
| 3 | 5000-44-16-9-7 | 2.0 | 3.3 |
| 4 | 5000-44-9-5-3 | 0.5 | 4.5 |
| 5 | 5000-44-9-5-3 | 1.0 | 5.5 |
| 6 | 5000-44-9-5-3 | 2.0 | 4.2 |

b) A model experiment for testing whether the polymeric micelle composed of the block copolymer and the polycation can gradually dissociate to decompose the micellar structure after being delivered to the target tissue or organ so as to increase the relaxivity in the target tissue or organ was then carried out.

To the polymeric micelle composed of PEG-P(Asp-ED-DTPA-Gd) 5000-44-16-7-4 and the polyallylamine having an average molecular weight of 15,000, NaCl solution having a concentration of 0.5M which was more than 3 times higher than that of the blood was added, and the mixture was left to stand at room temperature for 15 minutes, followed by subjecting the resulting mixture to gel permeation chromatography. As shown in Table 6 below, at any of the charge ratios of 0.5, 1.0 and 2.0, the elution volume before the addition of NaCl was within the range of 5.0 to 5.7 mL so that formation of polymeric micelle was indicated. On the other hand, after the addition of NaCl, the elution volume was increased to 10 to 11 mL. This indicates that the polymeric micellar structure was dissociated by the addition of NaCl. This fact also indicates that the micellar structure of the polymeric micelle according to the present invention is gradually dissociated mainly by the ions of NaCl in the body.

TABLE 6
Dissociation of Micellar Structure Formed of PEG-P(Asp-ED-DTPA-Gd)
and Polyallylamine (PAA) by Salt

| | | | Elution Volume (mL) in Gel | |
|---|---|---|---|---|
| | | Charge | Permeation Chromatography | |
| | Structure of | Ratio | Before | After |
| | PEG-P (Asp-ED- | —NH₂/ | Addition | Addition |
| Run | DTPA-Gd) (code) | —COOH | of NaCl | of NaCl |
| 1 | 5000-44-16-7-4 | 0.5 | 5.0 | 11 |
| 2 | 5000-44-16-7-4 | 1.0 | 5.7 | 10 |
| 3 | 5000-44-16-7-4 | 2.0 | 5.6 | 10 |

Table 7 below summarizes the change in relaxivity (R1) caused by the formation of polymeric micelle using each of the two types of polycations (polyallylamine and protamine) and PEG-P(Asp-ED-DTPA-Gd). The relaxivity (R1) is the value calculated by the Equation 1, and the larger the relaxivity (R1), the higher the ability to shorten the longitudinal relaxation time (T1) of water, and the higher the contrast in MRI images.

As shown by Run 1 in Table 7, by forming the polymeric micelle together with the polyallylamine having a molecular weight of 15,000, the relaxivity R1 was decreased by about 30% when compared with the case where the block copolymer exists as it is (that is, the state not forming the polymeric micelle). In Run 2 wherein the composition of the block copolymer was different, the relaxivity was largely changed due to the micelle formation. In Runs 3 and 4 where protamine which is a naturally occurring basic peptide was used as the polycation, a large change in the relaxivity was observed, and the relaxivity was decreased to about 1/15 in Run 3 and to ⅕ in Run 4 by the micelle formation. By these facts, the correctness of the basic design of the polymeric micelle MRI contrast agent with which the relaxivity may be largely changed due to the formation and dissociation of the polymeric micellar structure was proved.

$$\begin{matrix}Definition of Relaxivity R 1 & \\ \frac{1}{{T}_{1}} = \frac{1}{{T}_{1}^{0}} + {R}_{1} * [ Gd ] & Equation 1\end{matrix}$$

T₁: longitudinal relaxation time (s) of water in the presence of contrast agent
T₁⁰: longitudinal relaxation time (s) of water (in the absence of contrast agent)

R₁: relaxivity (mmol⁻¹·s⁻¹)

[Gd]: Concentration (mmol) of Gd atoms contained in contrast agent

TABLE 7
Change in Relaxivity (R1) by Polymeric Micelle Formation

| | | | Relaxivity (R1) | |
|---|---|---|---|---|
| | | | (mmol⁻¹ · s⁻¹) | |
| | | | | After |
| | Structure of | | Block | Polymeric |
| | PEG-P (Asp-ED- | | Copolymer | Micelle |
| Run | DTPA-Gd) (code) | Polycation | Alone | Formation |
| 1 | 12000-26-10-4-2 | Polyallylamine | 6.1 | 4.4 |
| 2 | 5000-44-9-7-15 | Polyallylamine | 9.3 | 1.2 |
| 3 | 12000-26-7-5-6 | Protamine | 6.5 | 0.42 |
| 4 | 5000-44-9-6-7 | Protamine | 18 | 3.6 |

### EXAMPLE 4

### Influence by Composition of Block Copolymer on Relaxivity R1

The influence by the composition of the block copolymer on the relaxivity R1 is summarized in Table 8. The relaxivity R1 was measured for each of the three types of PEG-P(Asp-ED-DTPA) with varying number of Gd atoms bound thereto under acidic condition at a pH of 2.8 to 4.8 or under neutral condition at a pH of 6.9 to 7.3. In any Run, the relaxivity R1 was smaller under the neutral condition than in the acidic condition. By changing the number of the bound Gd atoms using the same PEG-P(Asp-ED-DTPA), the larger the number of bound Gd atoms, the larger the relaxivity R1 (Runs 1-3, Runs 4-6, and Runs 7-9, respectively). By comparing Runs 1-3 and Runs 4-6, it was found that in Runs 4-6 wherein the number of ethylenediamine (ED) groups was smaller, the relaxivity R1 was larger. Further, by comparing Runs 4-6 and Runs 7-9 wherein the lengths of the polyethylene glycol chain were different, it was found that Runs 7-9 wherein the length of the polyethylene glycol chain was shorter exhibited higher relaxivity R1.

TABLE 8
Change in Relaxivity (R1) by Composition of Polymers

| | Structure of PEG-P | Relaxivity R1 (mmol⁻¹ · s⁻¹) | | | |
|---|---|---|---|---|---|
| | (Asp-ED-DTPA-Gd) | (volume in parentheses indicate pH) | | | |
| Run | (code) | Acidic Side | | Neutral Side | |
| 1 | 12000-26-10-4-6 | 12 | (3.8) | 6.8 | (6.9) |
| 2 | 12000-26-10-4-4 | 11 | (2.8) | 6.5 | (7.0) |
| 3 | 12000-26-10-4-3 | 5.5 | (4.8) | 4.0 | (7.0) |
| 4 | 12000-26-6-4-5 | 16 | (4.4) | 8.2 | (7.3) |
| 5 | 12000-26-6-4-4 | 10 | (3.8) | 7.7 | (7.2) |
| 6 | 12000-26-6-4-2 | 5.9 | (3.8) | 5.7 | (7.3) |
| 7 | 5000-26-5-4-4 | 16 | (3.6) | 14 | (7.2) |
| 8 | 5000-26-5-4-3 | 10 | (3.5) | 11 | (7.0) |
| 9 | 5000-26-5-4-2 | 6.7 | (4.2) | 7.3 | (7.0) |

### INDUSTRIAL AVAILABILITY

By the present invention, a contrast agent by which the T1-shortening ability of water in the blood vessels in the normal tissues and in the solid tumor tissue may be clearly distinguished is provided. Therefore, the present invention may be used in the field of production industry of the contrast agent and in the field of medical diagnosis which uses the contrast agent.

1. A contrast agent for magnetic resonance imaging, comprising as an effective ingredient a polymeric micelle having gadolinium (Gd) atoms in an inner core and an outer shell including hydrophilic polymer chain segments, which micelle is delivered to a tissue(s) and/or site(s) of solid tumor(s) in vivo, whose micellar structure is dissociated after being accumulated in said tissue(s) and/or site(s).

2. The contrast agent according to claim 1, wherein said polymeric micelle is formed of a block copolymer(s) having a hydrophilic polymer chain segment and a polymer chain segment having carboxyl groups and residues of a chelating agent(s) in its(their) side chains, gadolinium atoms coordinated to said block copolymer(s), and a polyamine(s).

3. The contrast agent according to claim 2, wherein said block copolymer is at least one selected from the group consisting of polyethylene glycol-block-poly(aspartic acid), polyethylene glycol-block-poly(glutamic acid), polyethylene glycol-block-poly(acrylic acid), polyethylene glycol-block-poly(methacrylic acid), poly(vinyl alcohol)-block-poly(aspartic acid), poly(vinyl alcohol)-block-poly(aspartic acid), poly(vinyl alcohol)-block-poly(glutamic acid), poly(vinyl alcohol)-block-poly(acrylic acid), poly(vinyl alcohol)-block-poly(methacrylic acid), poly(vinyl pyrrolidone)-block-poly(aspartic acid), poly(vinyl pyrrolidone)-block-poly(glutamic acid), poly(vinyl pyrrolidone)-block-poly(acrylic acid) and poly(vinyl pyrrolidone)-block-poly(methacrylic acid), to which residues of a chelating agent(s) are bound by covalent bond through carboxyl groups of said block copolymer and through linkers, as required.

4. The contrast agent according to claim 3, wherein said block copolymer is poly(ethylene glycol)-block-poly(aspartic acid) and said residues of a chelating agent(s) are introduced to 5% to 30% of the recurring units of the aspartic acid.

5. The contrast agent according to claim 1 or 2, wherein said block copolymer is at least one selected from the group consisting of those represented by the following formulae (1_{A-1}), (1_{A-2}), (1_{B-1}), (1_{B-2}), (1_{C-1}), (1_{C-2}), (1_{D-1}) and (1_{D-2}), respectively: 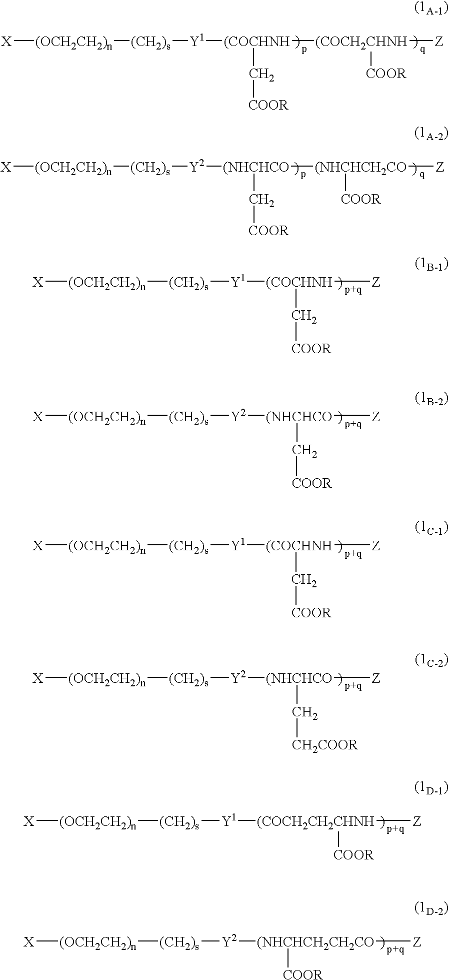
(in each of the above formulae, X represents hydrogen, C₁-C₆ alkyl, hydroxy-C₁-C₆ alkyl, acetalized or ketalized formyl-C₁-C₆ alkyl, amino-C₁-C₆ alkyl or benzyl;
Z represents hydrogen, hydroxy, C₁-C₆ alkyl, C₁-C₆ alkyloxy, phenyl-C₁-C₄ alkyl, phenyl-C₁-C₄ alkyloxy, C₁-C₄ alkylphenyl, C₁-C₄ alkylphenyloxy, C₁-C₆ alkoxycarbonyl, phenyl-C₁-C₄ alkyloxycarbonyl, C₁-C₆ alkylaminocarbonyl or phenyl-C₁-C₄ alkylaminocarbonyl;
n represents an integer of 10 to 10,000;
s represents an integer of 0 to 6;
OR represents OH, a linker or a residue of a linker-chelating agent, wherein the number of said residues of the chelating agent(s) is 5 to 30% of the total of p+q;
p and q independently represent integers of 1 to 300;
Y¹ represents —NH— or —R^{a}—(CH₂)ᵣ—R^{b}— wherein R^{a} represents OCO, OCONH, NHCO, NHCONH, COO or CONH, and R^{b} represents NH or O; and
Y² represents CO or —R^{c}—(CH₂)ᵣ—R^{d}— wherein R^{c} represents OCO, OCONH, NHCO, NHCONH, COO or CONH, R^{d} represents CO, and r represents an integer of 1 to 6).

6. The contrast agent according to claim 1 or 2, wherein said block copolymer is represented by the following Formula (2): 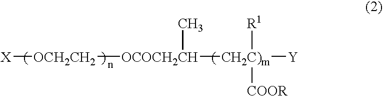
(wherein X represents hydrogen, C₁-C₆ alkyl, hydroxy-C₁-C₆ alkyl, acetalized or ketalized formyl-C₁-C₆ alkyl, amino-C₁-C₆ alkyl or benzyl;
R¹ represents hydrogen or methyl;
Y represents hydrogen, OH, Br, OR², CN, OCOR², NH₂, NHR² or N(R²)₂ (wherein R² represents hydrogen, C₁-C₆ alkyl, hydroxy-C₁-C₆ alkyl, acetalized or ketalized formyl-C₁-C₆ alkyl, amino-C₁-C₆ alkyl or benzyl);
m represents an integer of 4 to 600;
OR represents OH, a linker or a residue of a linker-chelating agent, wherein the number of said residues of the chelating agent(s) is 5 to 30% of the m).

7. The contrast agent according to claim 5 or 6, wherein said linker is NHCH₂CH₂NH₂ and said linker-chelating agent is —NHCH₂CH₂NHCOCH₂(HOOCH₂—)—NCH₂CH₂N(CH₂CH₂COOH)—CH₂CH₂N—(CHCHCOOH)₂.

8. The contrast agent according to any one of claims 2 to 7, wherein said chelating agent(s) is(are) at least one selected from the group consisting of diethylenetriaminepentaacetatic acid, tetraazacyclododecane and 1,4,7-tris(carboxymethyl)-10-(2′-hydroxypropyl)-1,4,7,10-tetraazacyclododecane.

9. The contrast agent according to any one of claims 2 to 8, wherein said polyamine is at least one selected from the group consisting of poly(L-lysine), poly(D-lysine), poly(L-arginine), poly(D-arginine), chitosan, spermine, spermidine, polyallylamine and protamine.

10. A block copolymer represented by the following Formula (1_{A-1}), (1_{A-2}), (1_{B-1}), (1_{B-2}), (1_{C-1}), (1_{C-2}), (1_{D-1}) or (1_{D-2}): 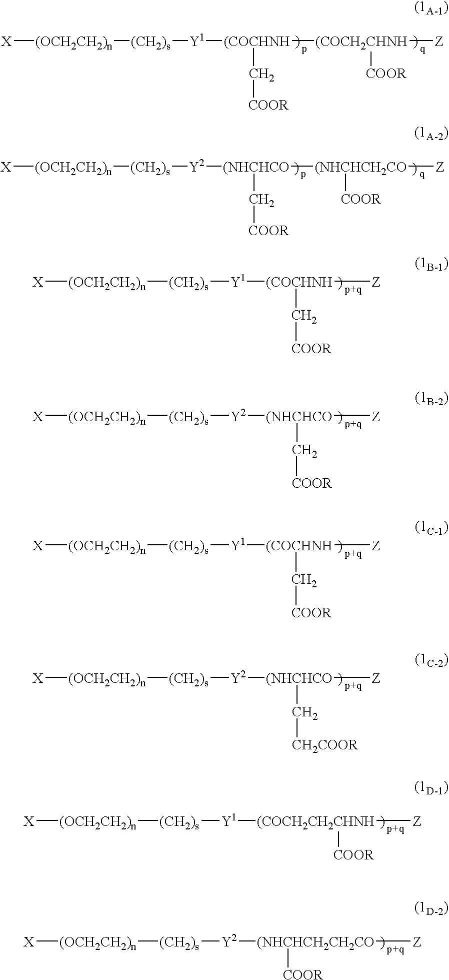
(in each of the above formulae, X represents hydrogen, C₁-C₆ alkyl, hydroxy-C₁-C₆ alkyl, acetalized or ketalized formyl-C₁-C₆ alkyl, amino-C₁-C₆ alkyl or benzyl;
Z represents hydrogen, hydroxy, C₁-C₆ alkyl, C₁-C₆ alkyloxy, phenyl-C₁-C₄ alkyl, phenyl-C₁-C₄ alkyloxy, C₁-C₄ alkylphenyl, C₁-C₄ alkylphenyloxy, C₁-C₆ alkoxycarbonyl, phenyl-C₁-C₄ alkyloxycarbonyl, C₁-C₆ alkylaminocarbonyl or phenyl-C₁-C₄ alkylaminocarbonyl;
n represents an integer of 10 to 10,000;
s represents an integer of 0 to 6;
OR represents OH, a linker or a residue of a linker-chelating agent, wherein the number of said residues of the chelating agent(s) is 5 to 30% of the total of p+q;
p and q independently represent integers of 1 to 300;
Y¹ represents —NH— or —R^{a}—(CH₂)ᵣ—R^{b}— wherein R^{a} represents OCO, OCONH, NHCO, NHCONH, COO or CONH, and R^{b} represents NH or O; and
Y² represents CO or —R^{c}—(CH₂)ᵣ—R^{d}— wherein R^{c} represents OCO, OCONH, NHCO, NHCONH, COO or CONH, R^{d} represents CO, and r represents an integer of 1 to 6).

11. A block copolymer represented by the following Formula (2): 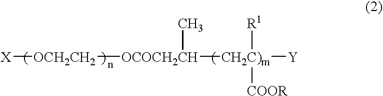
(wherein X represents hydrogen, C₁-C₆ alkyl, hydroxy-C₁-C₆ alkyl, acetalized or ketalized formyl-C₁-C₆ alkyl, amino-C₁-C₆ alkyl or benzyl;
R¹ represents hydrogen or methyl;
Y represents hydrogen, OH, Br, OR², CN, OCOR², NH₂, NHR² or N(R²)₂ (wherein R² represents hydrogen, C₁-C₆ alkyl, hydroxy-C₁-C₆ alkyl, acetalized or ketalized formyl-C₁-C₆ alkyl, amino-C₁-C₆ alkyl or benzyl);
m represents an integer of 4 to 600;
OR represents OH, a linker or a residue of a linker-chelating agent, wherein the number of said residues of the chelating agent(s) is 5 to 30% of the m).

12. The block copolymer according to claim 10 or 11, wherein said linker is NHCH₂CH₂NH₂ and said linker-chelating agent is —NHCH₂CH₂NHCOCH₂(HOOCH₂—)—NCH₂CH₂N(CH₂CH₂COOH)—CH₂CH₂N—(CHCHCOOH)₂.
